(19) United States
(12) Patent Application Publication
(10) Pub. No.: US 2015/0282975 A1
(43) Pub. Date: Oct. 8, 2015

(54) Orthopedic Cast

(71) Applicant: Lawrence D. Herzman, Farmingdale, NY (US)

(72) Inventor: Lawrence D. Herzman, Farmingdale, NY (US)

(21) Appl. No.: 14/246,824

(22) Filed: Apr. 7, 2014

(51) Int. Cl. A61F 5/05

(52) U.S. Cl. CPC A61F 5/05

(57) ABSTRACT

Aspects of the invention are directed to an apparatus comprising flexible tubing and a capsule. The flexible tubing is arranged in a matrix and encapsulates a first material. At the same time, the capsule is disposed within the flexible tubing, encapsulates a second material, and is breakable when subjected to at least one of impact and bending. The first material is segregated from the second material when the capsule is intact, but the first material mixes with the second material when the capsule is broken. Mixing of the first material and the second material forms a third material, the third material being substantially rigid. In one or more embodiments, the apparatus may be used as an orthopedic cast to stabilize and injury.

### FIELD OF THE INVENTION

The present invention relates generally to orthopedic devices and, more particularly, to medical casts for the treatment of bones and joints.

### BACKGROUND OF THE INVENTION

An orthopedic cast is commonly utilized to encase and stabilize broken bones to facilitate healing. A typical orthopedic cast consists of a cotton bandage that has been combined with Plaster of Paris (i.e., calcined gypsum). The Plaster of Paris slowly hardens after being exposed to water.

Nevertheless, despite their widespread use, conventional orthopedic casts suffer from several disadvantages. For example, it takes access to water to form an orthopedic cast, and, once a cast is placed, it may take up to 72 hours for the orthopedic cast to thoroughly dry and achieve full hardness. Moreover, a conventional orthopedic cast obscures the view of the underlying body portion, and does not allow the underlying portion to breathe or be cleaned. Skin underlying the orthopedic cast is therefore susceptible to skin-related complications including infections, rashes, and itching. Finally, conventional orthopedic casts may be heavy and unwieldy.

For at least the foregoing reasons, there is a need for improved orthopedic cast designs that address the above-described disadvantages while still effectively stabilizing the underlying injury.

### SUMMARY OF THE INVENTION

Embodiments of the present invention address the foregoing needs by providing improved orthopedic cast designs that address the above-identified deficiencies.

Aspects of the invention are directed to an apparatus comprising flexible tubing and a capsule. The flexible tubing is arranged in a matrix and encapsulates a first material. At the same time, the capsule is disposed within the flexible tubing, encapsulates a second material, and is breakable when subjected to at least one of impact and bending. The first material is segregated from the second material when the capsule is intact, but the first material mixes with the second material when the capsule is broken. Mixing of the first material and the second material forms a third material, the third material being substantially rigid.

Additional aspects of the invention are directed to a method for forming an orthopedic cast. Initially, flexible tubing and a capsule are received. The flexible tubing is arranged in a matrix and encapsulates a first material. At the same time, the capsule is disposed within the flexible tubing, encapsulates a second material, and is breakable when subjected to at least one of impact and bending. The first material is segregated from the second material when the capsule is intact. After receiving the flexible tubing and the capsule, the first material is mixed with the second material by breaking the capsule. Mixing the first material and the second material forms a third material, the third material being substantially rigid.

Advantageously, embodiments in accordance with aspects of the invention may: (1) be applied in the field without access to water; (2) be compactly carried by paramedics, firemen, policemen, soldiers, and the like in trauma bags or backpacks; (3) be tailored to conform to a particular patient's injury; and (4) act to effectively stabilize a patient's limb or neck without being unduly heavy and unwieldy. At the same time, once embodiments of the present invention are placed on a patient, the patient's underlying skin can be observed for blood circulation issues, rashes, and other trauma, and the underlying skin can be effectively cleaned to avoid skin-related complications.

### BRIEF DESCRIPTION OF THE DRAWINGS

These and other features, aspects, and advantages of the present invention will become better understood with regard to the following description, appended claims, and accompanying drawings where:

FIG. 1 shows a perspective view of an orthopedic cast in accordance with a first illustrative embodiment of the invention applied to a broken leg;

FIG. 2 shows a plan view of a portion of the FIG. 1 orthopedic cast;

FIG. 3 shows a sectional view of a portion of the FIG. 1 orthopedic cast along the plane indicated in FIG. 2;

FIG. 4 shows a perspective sectional view of a portion of the FIG. 1 orthopedic cast along the plane indicated in FIG. 2;

FIG. 5 shows an end-on sectional view of a portion of the FIG. 1 orthopedic cast;

FIG. 6 shows another end-on sectional view of a portion of the FIG. 1 orthopedic cast with a force applied;

FIG. 7 shows a plan view of two portions of flexible tubing joined by a clip in accordance with an illustrative embodiment of the invention;

FIG. 8 shows a sectional view of the FIG. 7 elements along the plane indicated in FIG. 7;

FIG. 9 shows a plan view of two portions of flexible tubing joined by an adhesive in accordance with an illustrative embodiment of the invention;

FIG. 10 shows a plan view of four portions of flexible tubing joined by welding in accordance with an illustrative embodiment of the invention;

FIG. 11 shows a perspective view of an orthopedic cast in accordance with a second illustrative embodiment of the invention applied to a broken arm; and

FIG. 12 shows a perspective view of an orthopedic cast in accordance with a third illustrative embodiment of the invention applied to an injured neck.

### DETAILED DESCRIPTION OF THE INVENTION

The present invention will be described with reference to illustrative embodiments. For this reason, numerous modifications can be made to these embodiments and the results will still come within the scope of the invention. No limitations with respect to the specific embodiments described herein are intended or should be inferred.

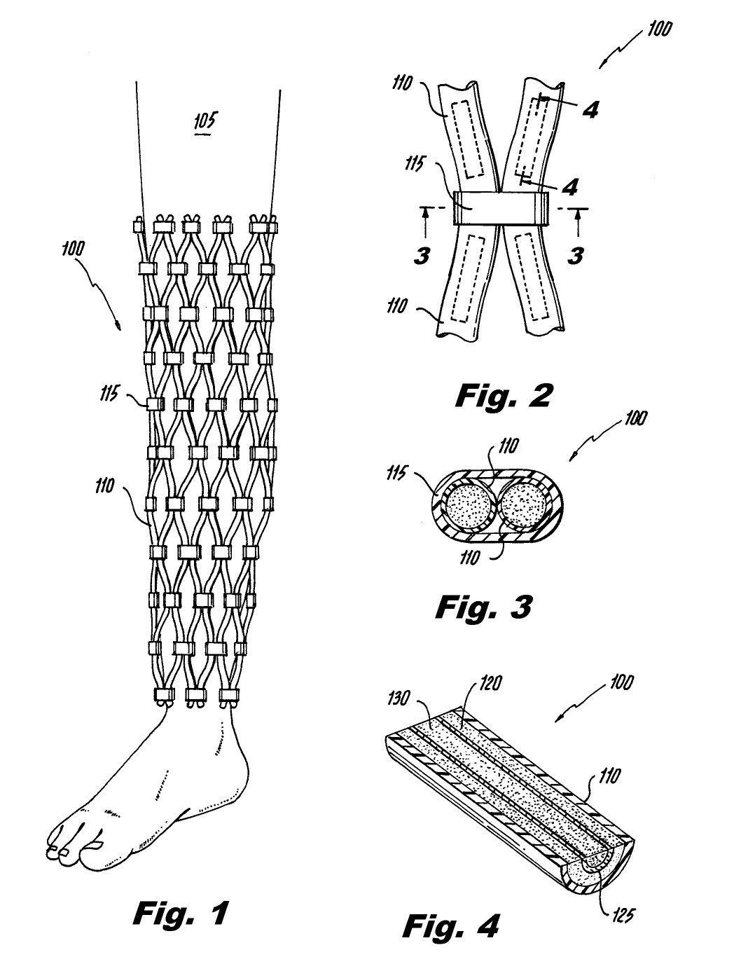

FIGS. 1-3 show an orthopedic cast 100 in accordance with a first illustrative embodiment of the invention. More particularly, FIG. 1 shows a perspective view of the orthopedic cast 100 applied to a broken leg 105, while FIG. 2 shows a plan view of a portion of the orthopedic cast 100, and FIG. 3 shows a sectional view of that portion of the orthopedic cast 100 along the plane indicated in FIG. 2. In this illustrative embodiment, portions of flexible tubing 110 are joined together with bands 115 to form a rigid matrix that encapsulates the broken leg 105. In so doing, the orthopedic cast 100 acts to stabilize the broken leg 105 so as to allow the broken leg 105 to properly heal or to remain stationary until further treatment becomes available.

Additional aspects of the orthopedic cast 100, before it is applied to a patient, are shown in FIGS. 4 and 5, where FIG. 4 shows a perspective sectional view of a portion of the orthopedic cast 100 along the plane indicated in FIG. 2, and FIG. 5 shows an end-on sectional view of that portion of the orthopedic cast 100. In accordance with aspects of the invention, the flexible tubing 110 encapsulates a first material 120. At the same time, the orthopedic cast 100 further comprises a plurality of capsules 125 that are disposed within the flexible tubing 110. The capsules 125 comprise a second material 130. With the capsules 125 intact, the first material 120 is segregated from the second material 130.

The capsules 125 are breakable when subjected to at least one of impact and bending. When a capsule 125 is broken, its contents (i.e., the second material 130) is allowed to mix with the first material 120. Such a condition is shown in the end-on sectional view in FIG. 6. Mixing of the first material 120 with the second material 130 forms a third material. The third material is substantially rigid. The forming of the third material from the mixing of the first material 120 and second material 130 is preferably relatively slow, taking, for example, several minutes to complete, during which time the orthopedic cast 100 remains relatively flexible.

When so configured, a method of utilizing the orthopedic cast 100 to stabilize the broken leg 105 in accordance with an embodiment of the invention comprises receiving the orthopedic cast 100 while the capsules 125 are still intact (FIGS. 4 and 5), and then causing the capsules 125 to break by applying a force or bending (FIG. 6). This initializes the mixing of the first material 120 and the second material 130 to form the third material. The orthopedic cast 100 may then be applied so as to surround the broken leg 105 (FIG. 1) while the forming of the third material is underway but still incomplete and the orthopedic cast 100 remains flexible. Once in place, the hardening of the orthopedic cast 100 may be allowed to continue until the orthopedic cast 100 becomes substantially rigid. The orthopedic cast 100 is thereby configured to fulfill its function of stabilizing the injury.

The orthopedic cast 100, when received and before use (i.e., with the capsules 125 intact), may be supplied in several forms. The orthopedic cast 100 may, for example, be supplied as a sleeve or sheet (folded, rolled, etc.). These various form factors allow the orthopedic cast 100 to be efficiently stored and transported in, for example, a trauma bag, military backpack, and the like. The orthopedic cast 100 may come in various sizes so as to encircle human arms, legs, and necks (as further detailed below). Where excess matrix remains after encircling a patient's limb or neck, that excess matrix may simply be gathered and made to overlap other portions of the matrix. Accordingly, it is contemplated that orthopedic casts in accordance with aspects of the invention may be supplied in three sizes appropriate for legs, arms, and necks, respectively, but that a large number of additional sizes may not be necessary.

The flexible tubing 110 may comprise any number of different polymeric formulations. For example, flexible tubing is readily available comprising polyurethane, nylon, polyethylene, polvinyl chloride, latex, silicone, silicon rubber, and polypropylene. One source of such tubing is United States Plastic Corp. (Lima, Ohio, USA). In contrast, the capsules 125 are preferably formed from a more brittle material so as to facilitate breaking by impact or bending. Suitable materials include, but are not limited to, glasses, ceramics, and polymers. Suitable polymers may include, but are not limited to, poly(methyl methacrylate) (PMMA) and polystyrene. In addition to using more brittle materials, the thickness of the capsules 125 may be minimized to aid in breakability. Nevertheless, for both the flexible tubing 110 and the capsules 125, it is emphasized that the specific materials recited herein are merely illustrative, and other equally suitable materials would also fall within the scope of the invention. These alternatives will be familiar to one having ordinary skill in the relevant arts. Reference is also made to C. Craver et al, Applied Polymer Science: 21st Century, Elsevier, 2000, which is hereby incorporated by reference herein.

At Standard Temperature and Pressure (STP; i.e., zero degrees Celsius temperature and one atmosphere pressure), the first material 120 and the second material 130 are preferably in liquid phases, while the third material, once formed, is primarily in a solid phase. In one or more embodiments, for example, the first material 120 and the second material 130 may comprise a liquid polymer resin and liquid curing agent that, when mixed, form a solid cured polymer. The third material may comprise, for example, cured epoxy, while the first material 120 and the second material 130 comprise a liquid epoxy resin and a liquid epoxy resin hardener, respectively. Alternatively, the first material 120 may comprise the epoxy resin hardener and the second material 130 may comprise the epoxy resin. In either configuration, the system chemically behaves similarly to a two-part epoxy coating or adhesive, that is, a chemical system wherein a liquid epoxy resin is mixed with a liquid epoxy resin hardener to form a solid cured epoxy.

A wide range of suitable epoxy resins and epoxy resin hardeners are available commercially, and these various materials would fall within the scope of the invention. Suitable classes of epoxy resins include, but are not limited to, bisphenol A epoxy resins, bisphenol F epoxy resins, novolac epoxy resins, and aliphatic epoxy resins. Suitable classes of epoxy resin hardeners include, but are not limited to, amines, acids, acid anhydrides, phenols, alcohols, and thiols. The rate at which an epoxy is cured may be tailored by choosing among the several different epoxy resin hardeners. Here again, aspects of forming epoxy materials by the mixing of two reactants will already be familiar to one skilled in the relevant arts. Reference is further made to J-P Pascault et al. (editor), Epoxy Polymers: New Materials and Innovations, John Wiley & Sons, 2009, which is also hereby incorporated by reference herein. Liquid epoxy resins and hardeners are available from, for example, US Composites, Inc. (West Palm Beach, Fla., USA).

At the same time, in alternative embodiments, other reactants may be used for the first material 120 and the second material 130. One of the two materials may comprise, as just a few more examples, liquid polystyrene resin, polyurethane resin, polyester resin, acrylic resin, and silicone resin. The remaining material may comprise a suitable hardener or curing agent (e.g., benzyol peroxide or methyl ethyl ketone peroxide).

While FIG. 1 shows the formation of the matrix by joining the flexible tubing 110 with the bands 115, the formation of the matrix can be accomplished in several ways. When joining flexible tubing, it is preferred that portions of the flexible tubing do not cross over other portions. This eliminates pressure points that might occur from such a configuration. Once the matrix of flexible tubing is formed (or at least partially formed), the first material and the capsules (with their second material) may be injected and inserted into the matrix.

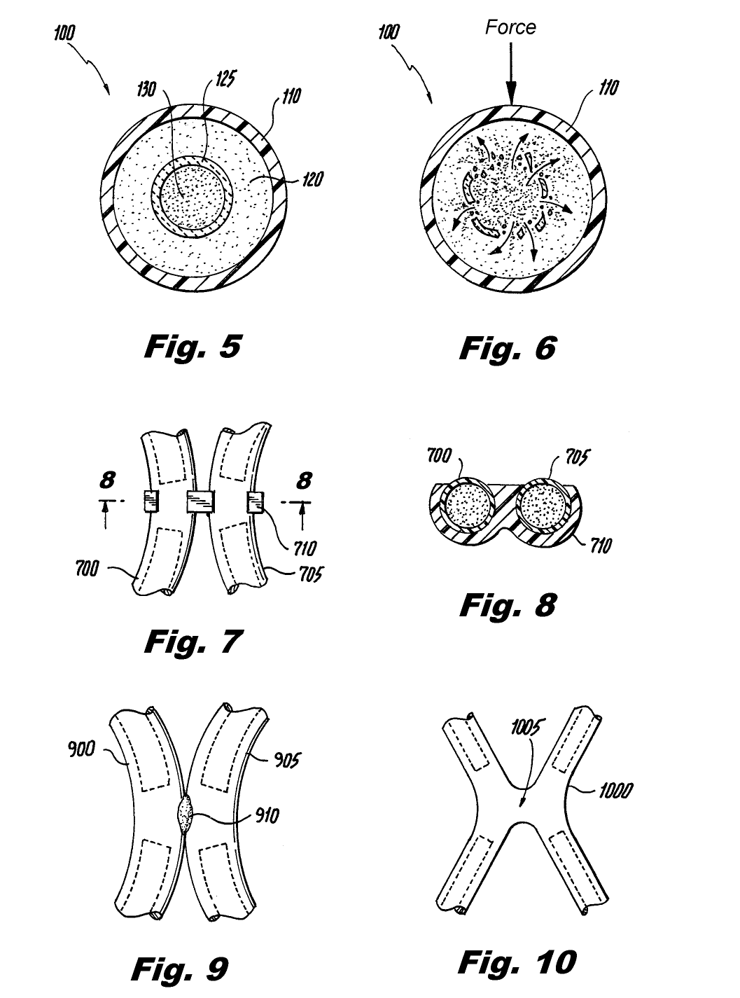

In one or more alternative embodiments, for example, portions of flexible tubing may be held together with clips to form the matrix. Use of a clip in this manner is shown in FIGS. 7 and 8, where FIG. 7 shows a plan view of a first portion of flexible tubing 700 joined to a second portion of flexible tubing 705 by a clip 710, and FIG. 8 shows a sectional view of the same elements along the plane indicated in FIG. 7. Advantageously, when bands or clips are utilized, the density of the matrix may be modified prior to or during application of an orthopedic cast to a patient. That is, the density of the matrix may be increased or decreased by manually moving and/or removing the bands or clips to change the spacing of the matrix. The resultant orthopedic cast may therefore be further tailored to a specific patient and/or injury.

In even one or more alternative embodiments, moreover, an adhesive may be used to join portions of flexible tubing. Such a configuration is illustrated in the top perspective view in FIG. 9, where a first portion of flexible tubing 900 is joined to a second portion of flexible tubing 905 using a wad of adhesive 910.

Lastly, in even one or more alternative embodiments, separate portions of the flexible tubing may be welded to others to form the matrix. This condition is shown in the plan view in FIG. 10, which shows four portions of flexible tubing 1000 welded together at a junction 1005. As will be familiar to a skilled artisan, welding of plastic tubing can be accomplished using several different welding techniques including, but not limited to, hot gas welding, heat sealing, hot plate welding, laser welding, ultrasonic welding, high frequency welding, and solvent welding. These techniques and others are described in D. A. Grewell et al., Plastics and Composites Welding Handbook, Volume 10, Hanser Gardener, 2003, which is hereby incorporated by reference herein.

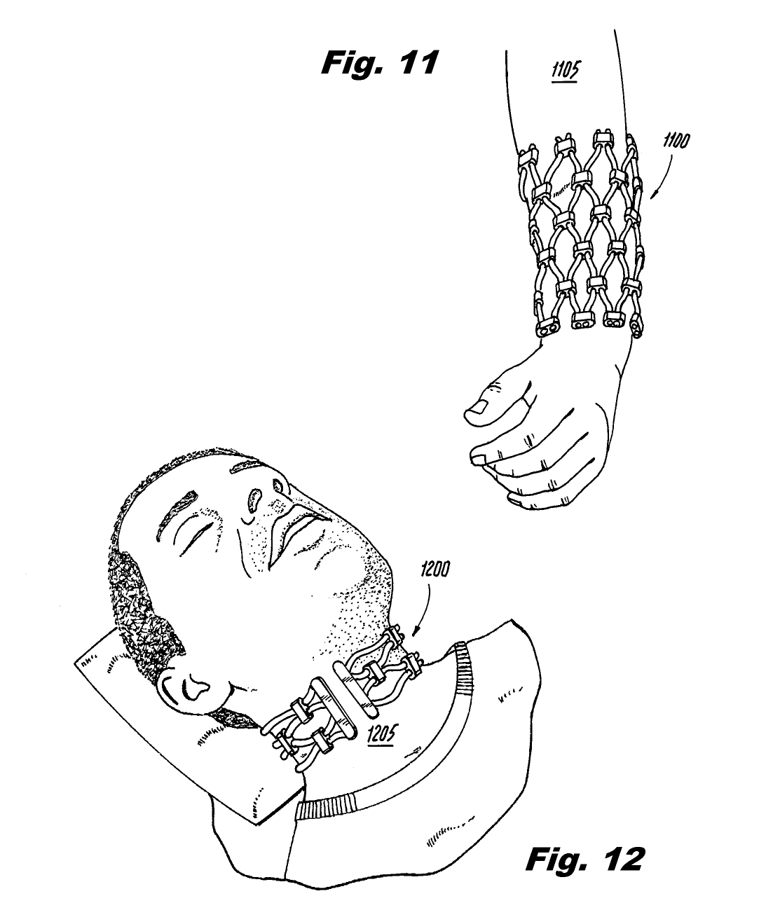

While FIG. 1 shows the utilization of the orthopedic cast 100 to stabilize a broken leg, it is emphasized that embodiments falling within the scope of the invention may be utilized for other purposes. FIG. 11, for instance, shows a perspective view of an orthopedic cast 1100 in accordance with a second illustrative embodiment of the invention positioned so as to stabilize a broken arm 1105. FIG. 12, moreover, shows a perspective view of an orthopedic cast 1200 in accordance with a third illustrative embodiment of the invention utilized to stabilize an injured neck 1205. When applied to the neck 1205, the orthopedic cast 1200 serves as a substitute for a conventional orthopedic neck collar. Nevertheless, unlike a conventional neck collar, the orthopedic cast 1200 allows continued access to the patient's neck in case a tracheotomy is required.

Based on the teachings presented herein, it will be recognized that the above-described embodiments and, more generally, embodiments falling within the scope of the invention, may provide several advantages when compared to conventional orthopedic casts (such as plaster casts and neck collars). Embodiments of the invention may variously, for example: (1) be applied in the field without access to water; (2) be compactly carried by paramedics, firemen, policemen, and soldiers in trauma bags or backpacks; (3) be tailored to conform to a particular patient or injury; and (4) effectively stabilize a patient's limb or neck without being unduly heavy and unwieldy. At the same time, once embodiments of the present invention are placed on a patient, the patient's underlying skin can be observed for blood circulation issues, rashes, and the like, and the underlying skin can be effectively cleaned to avoid skin-related complications.

It should again be emphasized that the above-described embodiments of the invention are intended to be illustrative only. Other embodiments can use different types and arrangements of elements for implementing the described functionality. For example, while particular shapes of orthopedic casts are described above, other orthopedic casts falling within the scope of the invention may have very different shapes and sizes. Alternative embodiments of the invention may, for example, be sized to encircle an entire leg (e.g., from torso to foot) or an entire arm (e.g., from shoulder to wrist). Even more, alternative embodiments may be dimensioned to stabilize a foot or hand. These numerous alternative embodiments within the scope of the appended claims will be apparent to one skilled in the art.

Moreover, all the features disclosed herein may be replaced by alternative features serving the same, equivalent, or similar purposes, unless expressly stated otherwise. Thus, unless expressly stated otherwise, each feature disclosed is one example only of a generic series of equivalent or similar features.

Any element in a claim that does not explicitly state “means for” performing a specified function or “step for” performing a specified function is not to be interpreted as a “means for” or “step for” clause as specified in 35 U.S.C. §112, ¶6. In particular, the use of “step of” in the claims herein is not intended to invoke the provisions of 35 U.S.C. §112, ¶6.

What is claimed is:

1. An apparatus comprising:
flexible tubing, the flexible tubing arranged in a matrix and encapsulating a first material; and
a capsule, the capsule disposed within the flexible tubing, encapsulating a second material, and breakable when subjected to at least one of impact and bending;
wherein:
the first material is segregated from the second material when the capsule is intact;
the first material mixes with the second material when the capsule is broken; and
mixing of the first material and the second material forms a third material, the third material being substantially rigid.

2. The apparatus of claim 1, wherein the flexible tubing comprises a material selected from the group consisting of polyurethane, nylon, polyethylene, polyvinyl chloride, latex, silicon rubber, and polypropylene.

3. The apparatus of claim 1, wherein the capsule is more brittle than the flexible tubing.

4. The apparatus of claim 1, wherein the apparatus defines a sleeve.

5. The apparatus of claim 1, wherein a first portion of the flexible tubing is welded to a second portion of the flexible tubing.

6. The apparatus of claim 1, further comprising a clip, the clip fixating a first portion of the flexible tubing to a second portion of the flexible tubing.

7. The apparatus of claim 1, further comprising a band, the band fixating a first portion of the flexible tubing to a second portion of the flexible tubing.

8. The apparatus of claim 1, further comprising an adhesive, the adhesive fixating a first portion of the flexible tubing to a second portion of the flexible tubing.

9. The apparatus of claim 1, further comprising a fastener, the fastener defining four slots, each of the four slots receiving a different portion of flexible tubing.

10. The apparatus of claim 1, wherein the capsule comprises a material selected from the group consisting of glass, ceramic, and polymer.

11. The apparatus of claim 1, wherein, under standard pressure and temperature conditions:
the first material is a liquid;
the second material is a liquid; and
the third material is a solid.

12. The apparatus of claim 1, wherein:
the first material comprises a polymer resin;
the second material comprises a curing agent; and
the third material comprises a cured polymer.

13. The apparatus of claim 1, wherein:
the first material comprises a curing agent;
the second material comprises a polymer resin; and
the third material comprises a cured polymer.

14. The apparatus of claim 1, wherein:
the first material comprises an epoxy resin;
the second material comprises an epoxy hardener; and
the third material comprises a cured epoxy.

15. The apparatus of claim 1, wherein:
the first material comprises an epoxy hardener;
the second material comprises an epoxy resin; and
the third material comprises a cured epoxy.

16. A method comprising the steps of:
receiving flexible tubing, the flexible tubing arranged in a matrix and encapsulating a first material;
receiving a capsule, the capsule disposed within the flexible tubing, encapsulating a second material, and breakable when subjected to at least one of impact and bending; and
causing the first material to mix with the second material by breaking the capsule;
wherein:
the first material is segregated from the second material when the capsule is intact; and
mixing of the first material and the second material forms a third material, the third material being substantially rigid.

17. The method of claim 16, further comprising the step of causing the matrix to surround a portion of a human being after causing the first material to mix with the second material.
